(19) United States
(12) Patent Application Publication
(10) Pub. No.: US 2018/0273442 A1
(43) Pub. Date: Sep. 27, 2018

(54) Methane Conversion

(71) Applicant: ExxonMobil Chemical Patents Inc., Baytown, TX (US)

(72) Inventors: Teng Xu, Houston, TX (US); Yi Liu, Beijing (CN); Tianyun Wang, Beijing (CN); Yi Zhang, Beijing (CN)

(21) Appl. No.: 15/916,389

(22) Filed: Mar. 9, 2018

Related U.S. Application Data
(60) Provisional application No. 62/476,948, filed on Mar. 27, 2017.

(51) Int. Cl. C07C 2/86; B01J 29/48; C07C 9/14; C07C 9/04; C07C 15/08; C07C 31/04

(52) U.S. Cl. CPC C07C 2/864; B01J 29/48; C07C 9/14; C07C 9/04; C07C 2529/40; C07C 31/04; C10G 2300/1081; C10G 2400/30; C07C 15/08

(57) ABSTRACT

This disclosure relates to the conversion of methane to hydrocarbon of greater molecular weight, including aromatic hydrocarbon such as xylenes, to materials and equipment useful in such conversion, and to the use of such conversion for, e.g., natural gas upgrading.

### PRIORITY CLAIM

This application claims priority to and the benefit of U.S. Patent Application Ser. No. 62/476,948 filed Mar. 27, 2017, which is incorporated by reference herein in its entirety.

### FIELD OF THE INVENTION

This disclosure relates to the conversion of methane to hydrocarbon of greater molecular weight, including aromatic hydrocarbon such as xylenes, to materials and equipment useful in such conversion, and to the use of such conversion for, e.g., natural gas upgrading.

### BACKGROUND OF THE INVENTION

Although methane is abundant, its relative inertness has limited its utility in conversion processes for producing higher-value hydrocarbon. For example, oxidative coupling methods generally involve highly exothermic and potentially hazardous methane combustion reactions, typically require expensive oxygen generation facilities, and produce large quantities of environmentally-sensitive carbon oxides. In addition, non-oxidative methane aromatization is equilibrium-limited, and a temperature ≥1200° C. is typically needed to achieve appreciable methane conversion and appreciable yield of aromatic hydrocarbon.

To obviate this problem, catalytic processes have been proposed for converting methane with appreciable aromatic hydrocarbon yield at a lesser temperature, e.g., as disclosed in Wang, L., Tao, L., Xie, M. et al. Catalysis Letters, Vol. 21, Issue 1, pp. 35-41 (1993). Other references disclose improving aromatic hydrocarbon yield by carrying out the catalytic methane conversion in the presence of at least one co-reactant. For example, U.S. Pat. No. 5,936,135 discloses reacting methane at a temperature in the range of 300° C. to 600° C. with (i) a C₂₋₁₀ olefin, and/or (ii) a C₂₋₁₀ paraffin in the presence of a bifunctional pentasil zeolite catalyst.

Other processes utilize organic oxygenate as a co-reactant for the non-oxidative methane conversion to produce higher hydrocarbon, including aromatics. For example, U.S. Pat. No. 7,022,888 discloses a process for the non-oxidative conversion of methane simultaneously with the conversion of an organic oxygenate, represented by a general formula: CnH₂n+1OCmH₂m+1, wherein C, H and O are carbon, hydrogen and oxygen, respectively; n is an integer having a value between 1 and 4; and m is an integer having a value between zero and 4. The methane and oxygenate are converted to C₂₊ hydrocarbon, particularly to gasoline range C₆-C₁₀ hydrocarbon and hydrogen, using a bifunctional pentasil zeolite catalyst.

One difficulty encountered with these conventional approaches to methane aromatization results from an appreciable yield of undesirable by-products such as coke, which accumulates on the catalyst and shortens catalyst lifetime. In order to at least partially overcome this difficulty a recent reference, Y. Liu, D. Li, T. Wang, Y. Liu, T. Xu and Y. Zhang, ACS Catalysis, Vol. 6, pp. 5366-5370 (2016), discloses the co-conversion of methane and methanol over a Mo-impregnated H-ZSM-5 catalyst. The reference reports a methane conversion >25%, a selectivity to benzene, toluene, and xylenes (collectively, “BTX”) >90%. Even though the conversion is carried out at a temperature of 700° C., the reference reports a steady-state run length of sixty hours.

Although run lengths on the order of sixty hours represents an improvement over earlier work, further decreases in coke yield and further increases in run length are needed to achieve a commercially practical methane conversion process featuring appreciable methane conversion and appreciable aromatic hydrocarbon yield over a wide range of process conditions.

### SUMMARY OF THE INVENTION

The invention is based in part on the discovery that when methane is reacted with a particular class of co-feeds in the presence of a particular class of molecular sieve catalysts, coke yield is much less than in the case of conventional catalytic methane conversion reactions operating under similar process conditions. Moreover, methane conversion and yields of desired products, such as C₅₊ hydrocarbon, particularly BTX yield, are typically the same as or better than those of conventional processes operating under similar process conditions.

Accordingly, certain aspects of the invention relate to a process for producing C₅₊ hydrocarbon from methane and at least one co-feed comprising one or more of C₂₊ hydrocarbon, C₁₊ organic oxygenate, and inorganic oxygenate. The feed and co-feed react in the presence of a conversion catalyst comprising at least one molecular sieve and at least one active metal, which can be in the form, e.g., of one or more metal carbides and/or one or more metal oxycarbides. The molecular sieve has a framework of interconnected atoms, an outer surface, and a plurality of pores having an average pore size of 4 Å to 7 Å. Framework can include metal atoms e.g., aluminum atoms. The active metal is a metal selected from those of Groups 3 to 13 of the Periodic Table. A feature of the conversion catalyst is that ≥90 wt. % of the active metal is located in the pores, with ≤10 wt. % being proximate to the outer surface. Another feature is that the conversion catalyst comprises ≤10 wt. % of metal in any form other than (A) framework metal if any and (B) the active metal. The process includes contacting the methane and the co-feed with the conversion catalyst under conversion conditions which include a temperature <1200° C. to convert at least part of the methane and co-feed to a product comprising at least 5 wt. % of C₅₊ hydrocarbon based on the weight of the product. At least a portion of the C₅₊ hydrocarbon can be separated from the product and conducted away.

In other aspects, the invention relates to producing C₅₊ hydrocarbon from a methane-containing natural gas in the presence of the specified conversion catalyst. In these aspects, the co-feed is in the form of one or more C₂₊ hydrocarbon compounds that are present in the natural gas with the methane.

In still other aspects, the invention relates to a conversion product produced by catalytically converting methane and at least one co-feed comprising one or more of C₂₊ hydrocarbon, C₁₊ organic oxygenate, and inorganic oxygenate. The conversion includes contacting the methane and co-feed with a conversion catalyst produced from a synthesis mixture. The synthesis mixture comprises at least one aluminum source, water, a templating agent, and one silica source, wherein (i) ≥90 wt. % of the silica source comprises SiO₂ impregnated with at least one metal, or compound thereof, selected from Groups 3 to 13 of the Periodic Table and (ii) the aluminum source, the water, the templating agent, and any other components of the synthesis mixture together comprise ≤0.1 wt. % of oxide of silicon.

The synthesis mixture is reacted under hydrothermal reaction conditions to produce a reaction product comprising molecular sieve. At least a portion of the molecular sieve is activated to produce the conversion catalyst. The methane and co-feed contact the conversion catalyst under conversion conditions which include a temperature <1200° C. to convert at least part of the methane and co-feed to the conversion product, the conversion product comprising at least 5 wt. % of C₅₊ hydrocarbon. In further aspects, the invention relates to (i) systems and apparatus that are useful for carrying out any of the preceding aspects, (ii) the specified conversion catalyst, and (iii) the specified conversion products.

### DETAILED DESCRIPTION

### Definitions

For the purpose of this description and appended claims the following terms are defined.

The term “Cₙ” hydrocarbon means hydrocarbon having n carbon atom(s) per molecule, wherein n is a positive integer. The term “Cₙ₊” hydrocarbon means hydrocarbon having at least n carbon atom(s) per molecule. The term “Cₙ₋” hydrocarbon means hydrocarbon having no more than n carbon atom(s) per molecule. The term “hydrocarbon” means a class of compounds containing hydrogen bound to carbon, and encompasses (i) saturated hydrocarbon, (ii) unsaturated hydrocarbon, and (iii) mixtures of hydrocarbons, and including mixtures of hydrocarbon compounds (saturated and/or unsaturated), such as mixtures of hydrocarbon compounds having different values of n.

The terms “alkane” and “paraffinic hydrocarbon” mean substantially-saturated compounds containing hydrogen and carbon only, e.g., those containing ≤1% (molar basis) of unsaturated carbon atoms. As an example, the term alkane encompasses C₂ to C₂₀ linear, iso, and cyclo-alkanes. Aliphatic hydrocarbon means hydrocarbon that is substantially free of hydrocarbon compounds having carbon atoms arranged in one or more rings.

The term “unsaturate” and “unsaturated hydrocarbon” refer to one or more C₂₊ hydrocarbon compounds which contain at least one carbon atom directly bound to another carbon atom by a double or triple bond. The term “olefin” refers to one or more unsaturated hydrocarbon compound containing at least one carbon atom directly bound to another carbon atom by a double bond. In other words, an olefin is a compound which contains at least one pair of carbon atoms, where the first and second carbon atoms of the pair are directly linked by a double bond. The term “aromatics” and “aromatic hydrocarbon” mean hydrocarbon compounds containing at least one aromatic ring. Non-aromatic hydrocarbon is hydrocarbon comprising ≤1 wt. % of carbon atoms included in aromatic rings.

The term “oxygenate” means a class of compounds which include at least one oxygen atom, e.g., alcohol and ether. The term “alcohol” means a class of oxygenate compounds which include at least one aliphatic carbon bound to a hydroxyl group, but excluding other oxygenate compounds such as aldehyde, ketone, and carboxylic acid. The term “Cₙ” alcohol means alcohol having n carbon atom(s) per molecule, wherein n is a positive integer. The term “Cₙ₊” alcohol means alcohol having at least n carbon atom(s) per molecule, wherein n is a positive integer. The term “Cₙ₋” alcohol means alcohol having no more than n number of carbon atom(s) per molecule, wherein n is a positive integer. The term alcohol encompasses (i) saturated and unsaturated alcohol, (ii) alcohol having one hydroxyl group per alcohol molecule (mono-alcohol) and alcohol having a plurality of hydroxyl groups per alcohol molecule (di-alcohol, tri-alcohol, etc.), (iii) primary, secondary, and tertiary alcohol, (iv) alcohol having a terminal hydroxyl group (1-alcohol) and alcohol having a hydroxyl group in a non-terminal position (2-alcohol, 3-alcohol, etc.), and (v) mixtures of two or more alcohol compounds, including mixtures of alcohol compounds having different values of n.

The term “active metal” means one or more metals that are (i) selected from those of Groups 3 to 13 of the Periodic Table, and (ii) not part of the molecular sieve's framework. A feature of the conversion catalyst is that ≤10 wt. % of the active metal is proximate to the molecular sieve's outer surface. In this context, “proximate to” means within about 10 Å of the outer surface, e.g., within 5 Å, such as within 2.5 Å. The term “carbidic metal” means one or more metals in carbidic form, e.g., in forms such as one or more carbides of such metal or metals and/or one or more oxycarbides of such metal or metals. The metal or metals included in the carbidic forms are selected from metals of Groups 3 to 13 of the Periodic Table.

The term “reaction zone” or “reactor zone” mean a location within a reactor, e.g., a specific volume within a reactor, for carrying out a specified reaction. A reactor or reaction stage can encompass one or more reaction zones. More than one reaction can be carried out in a reactor, reactor stage, or reaction zone. For example, a reaction stage can include a first zone for carrying out first and second reactions and a second zone for carrying out a third reaction, where the first reaction (e.g., dehydrogenation) can be the same as or different from the second reaction, and the third reaction (e.g., CO₂ methanation) can be the same as or different from the second reaction.

The term “selectivity” refers to the production (on a weight basis) of a specified compound in a catalytic reaction. As an example, the phrase “a light hydrocarbon conversion reaction has a 100% selectivity for aromatic hydrocarbon” means that 100% of the light hydrocarbon (weight basis) that is converted in the reaction is converted to aromatic hydrocarbon. When used in connection with a specified reactant, the term “conversion” means the amount of the reactant (weight basis) consumed in the reaction. For example, when the specified reactant is C₄ paraffinic hydrocarbon, 100% conversion means 100% of the C₄ paraffinic hydrocarbon is consumed in the reaction. Yield (weight basis) is conversion times selectivity.

The term “Periodic Table” means the Periodic Chart of the Elements, as it appears on the inside cover of The Merck Index, Twelfth Edition, Merck & Co., Inc., 1996.

Certain aspects of the invention relate to reacting methane and one or more of the specified co-feeds in the presence of the specified conversion catalyst. To produce C₅₊ hydrocarbon, particularly aromatics, and more particularly BTX. Representative sources of methane and the co-feeds will now be described in more detail. The invention is not limited to these sources, and this description is not meant to foreclose other sources of methane and co-feeds within the broader scope of the invention.

### Feeds

In certain aspects, a combined feed is reacted in the presence of the specified conversion catalyst. In these aspects, the combined feed comprises the desired amounts of methane and co-feed, and desired relative amounts of co-feed constants. The combined feed is typically conducted to a reactor containing a catalytically effective amount of the specified conversion catalyst. At least a portion of the methane and the co-feed in the combined feed are converted in the presence of the specified conversion catalyst under methanol conversion conditions to C₅₊ hydrocarbon, e.g., aromatic hydrocarbon, such as BTX. The co-feed is typically a co-reactant, in the sense that it reacts with the methanol during the methanol conversion reaction. For example, in certain aspects atoms originating in co-feed constituents, e.g., carbon atoms and/or hydrogen atoms of methanol in the co-feed, are included in one or more of the C₅₊ hydrocarbon compounds in the conversion product, e.g., by way of a chemical bond. Typically, at least a portion of the combined feed is in the vapor phase during the conversion. For example, ≥75.0 wt. %, e.g., ≥90.0 wt. %, such as ≥99.0 wt. % of the combined feed can be in the vapor phase during the conversion.

Suitable combined feeds include those which comprise 40 mole % to 80 mole % of methane, with ≥90 wt. % of the combined feed comprising at least one co-feed such as methanol. The remainder of the combined feed, if any, can comprise diluent, for example. The term “diluent” in this context means species which do not react in significant amounts with methane and/or co-feed to produce the C₅₊ hydrocarbon under the specified conversion conditions. Suitable diluent includes molecular nitrogen and one or more of the noble gases. In certain aspects, the combined feed comprises diluent in an amount in the range of from 0.1 mole % to 50 mole %, per mole of combined feed. Where present, some or all of the diluent can be present as by-products of the process used to produce the combined feed's methane and/or co-feed.

The methane and/or the co-feed (and even certain diluents) can be obtained from natural and/or synthetic sources. For example, the combined feed can be obtained from natural hydrocarbon sources including those associated with producing petroleum. Synthetic hydrocarbon sources include, e.g., streams obtained from refining and petrochemical plants, e.g., acetylene and/or olefin co-feed sources from one or more steam crackers. Synthetic hydrocarbon sources also include those in which hydrocarbon within a geological formation has been purposefully subjected to one or more chemical transformations. Synthetic hydrocarbon sources also include process recycle streams, e.g., a portion of the product obtained from the methane conversion reaction. Such recycle, when used, can include methane and/or co-feed.

Natural and/or synthetic hydrocarbon having 2 or more carbon atoms (C₂₊ hydrocarbon) is a suitable co-feed, e.g., C₂₊ aliphatic hydrocarbon, such as C₂₊ paraffin hydrocarbon, and particularly C₂ to C₉ paraffinic hydrocarbon. For example, the co-feed can comprises C₂ to C₅ alkane, such as ≥50.0 wt. % of C₂ to C₅ alkane, or ≥75.0 wt. %, or ≥90.0 wt. %, or ≥99.0 wt. %.

In certain aspects, e.g., those utilizing a co-feed comprising C₂₊ non-aromatic hydrocarbon, the combined feed source can include natural gas, e.g., can consist essentially of or even consist of natural gas. Natural gas is (i) a mixture comprising hydrocarbon, (ii) primarily in the vapor phase at a temperature of 15° C. and a pressure of 1.013 bar (absolute), and (iii) withdrawn from a geologic formation. Natural gas can be obtained, e.g., from one or more of petroleum deposits, coal deposits, and shale deposits. Natural gas produced by conventional production methods is suitable, but the invention is not limited thereto. The natural gas can be a raw gas, namely one that is obtained from a geologic formation without intervening processing (such as fractionation with reflux), except for treatments to (i) adjust the amount of CO₂ in the feed, (ii) remove impurities such as water and/or any other liquids, mercaptans, hydrogen sulfide, carbon dioxide; and (iii) adjust the relative amounts of non-aromatic hydrocarbon in the feed (typically by separating C₄₊ hydrocarbon in one or more vapor-liquid separators). Conventional methods can be used for removing impurities and/or adjusting the relative amount of the non-aromatic hydrocarbon compounds present in the feed, but the invention is not limited thereto. For example, certain components in the natural gas can be liquefied by exposing the natural gas to a temperature in the range of −57° C. to 15° C., e.g., −46° C. to 5° C., such as −35° C. to −5° C. At least a portion of the liquid phase can be separated in one or more vapor-liquid separators, e.g., one or more flash drums. One suitable raw natural gas has a non-aromatic hydrocarbon component comprising 3 mole % to 70 mole % methane, and a co-feed comprising 10 mole % to 50 mole % ethane, 10 mole % to 40 mole % propane, and 5 mole % to 40 mole % butanes and 1 mole % to 10 mole % of total C₅ to C₉ hydrocarbon. One or more of the C₂₊ hydrocarbon in the natural gas can be utilized as the sole co-feed. In other aspects, the co-feed comprises one or more of the C₂₊ hydrocarbons in the natural gas and additionally one or more of (i) C₂₊ hydrocarbon obtained from sources other than natural gas, (ii) C₁₊ organic oxygenate, and (iii) inorganic oxygenate. One suitable combined feed includes natural gas comprising methane and ≥1 wt. % of C₂₊ non-aromatic hydrocarbon and ≥0.005 wt. % of CO₂, such as raw natural gas, e.g., ≥75 wt. %, or ≥90 wt. %, or ≥95 wt. %.

When the source of methane and/or co-feed is raw gas, any form of raw gas can be used, although those containing an appreciable amount of CO₂ (e.g., ≥0.05 wt. % of CO₂, such as ≥0.5 wt. %, or ≥1 wt. %, or ≥5 wt. %) are particularly useful. The raw gas can be, e.g., one or more of (i) gas obtained from a natural gas well (“Gas Well”, Non-associated”, or “Dry” gas), (ii) natural gas obtained from a condensate well (“Condensate Well Gas”), and (iii) casing head gas (“Wet” or “Associated” gas). Table 1 includes typical raw gas compositional ranges (mole %) and, parenthetically, typical average composition (mole %) of certain raw gasses.

TABLE 1

| | Associated | | | | Condensate | |
|---|---|---|---|---|---|---|
| Component | Gas | | Dry Gas | | Well Gas | |
| CO₂ | 0-50 | (0.63) | 0-25 | (0) | 0-25 | (0) |
| N₂ | 0-50 | (3.73) | 0-25 | (1.25) | 0-25 | (0.53) |
| H₂S | 0-5 | (0.57) | 0-5 | (0) | 0-5 | (0) |
| CH₄ | 0-80 | (64.48) | 0-97 | (91.01) | 0-98 | (94.87) |
| C₂H₆ | 5-20 | (11.98) | 2-10 | (4.88) | 1-5 | (2.89) |
| C₃H₈ | 2-10 | (8.75) | 0.5-5 | (1.69) | 0.1-5 | (0.92) |
| i-butane | 0.1-5 | (0.93) | 0.05-1 | (0.14) | 0.1-5 | (0.31) |
| n-butane | 1-5 | (2.91) | 0.05-2 | (0.52) | 0.05-2 | (0.22) |
| i-pentane | 0.05-2 | (0.54) | 0.01-1 | (0.09) | 0.1-1 | (0.09) |

In certain aspects, ≥50 wt. % of the co-feed comprises C₁₊ organic oxygenate, e.g., one or more members of a class of compounds represented by a general formula: CnH₂n₊₁OCmH₂m₊₁, wherein C, H and O are carbon, hydrogen and oxygen, respectively. Typically, n is an integer having a value from 1 to 4, e.g., from 1 to 3, such as 1 or 2; and m is an integer having a value from zero to 3, e.g., from zero to 2, such as zero or 1. Examples of suitable C₁₊ organic oxygenate include methanol, ethanol, dimethyl ether, diethyl ether, dipropyl ether, dibutyl ether, methyl ethyl, ether, methyl propyl ethers, methyl butyl ethers, and mixture thereof. Preferred C₁₊ organic oxygenate includes methanol, ethanol, dimethyl ether, diethyl ether, and mixtures thereof. In certain aspects, the C₁₊ organic oxygenate comprises ≥90.0 wt. % of C₁ to C₄ alcohol and/or C₂ to C₈ dialkyl ether, based on the weight of the C₁₊ organic oxygenate, e.g., ≥90.0 wt. % of methanol, ethanol, dimethyl ether, diethyl ether and mixtures thereof. When the co-feed comprises C₁₊ organic oxygenate, e.g., organic alcohol, such as methanol, the methane: C₁₊ oxygenate weight ratio in the combined feed is typically in the range of from 0.5 to 60, e.g., 1 to 50, such as 5 to 20. For example, when the alcohol is methanol, the methane:methanol molar ratio is typically in the range of from 2 to 100, e.g., from 5 to 50, such as from 10 to 30.

Alternatively or in addition to C₂₊ hydrocarbon and C₁₊ organic oxygenate, the co-feed can comprise inorganic oxygenate, e.g., ≥50 wt. % of C₁₊ inorganic oxygenate, such as ≥75 wt. %, or ≥90 wt. %. For example, suitable combined feeds can include those which comprise ≥9 mole % of methane, e.g., ≥25 mole %, such ≥40 mole %, wherein the molar ratio of methane to C₁₊ inorganic oxygenate in the combined feed is in the range of from 0.6:1 to 20:1, such as from 5:1 to 15:1, or from 7:1 to 10:1. The mole percents are based on per mole of combined feed. The C₁₊ inorganic oxygenate can comprise, e.g., one or more of CO, CO₂ and formaldehyde. For example, the C₁₊ inorganic oxygenate can comprise ≥50.0 wt. % of CO, based on the weight of the C₁₊ inorganic oxygenate, such as ≥75.0 wt. %, or ≥90.0 wt. %, or ≥99.0 wt. %. The C₁₊ inorganic oxygenate is substantially all CO. Optionally, balance of C₁₊ inorganic oxygenate, if any, can be CO₂. Particularly in aspects where the co-feed includes inorganic oxygenate, the co-feed can optionally further comprise molecular hydrogen. For example, one suitable co-feed has a molecular hydrogen: C₁₊ inorganic oxygenate molar ratio ≥0.6, e.g., ≥1.0, such as ≥10.0, or in the range of from 0.5:1 to 20:1, e.g., 0.6:1 to 20:1. Syngas is an example of one such co-feed, e.g., a syngas having a molecular hydrogen: (CO+CO₂) molar ratio ≥0.6, e.g., ≥1.0, such as ≥10.0, or in the range of from about 0.6 to about 20. Optionally, the molecular hydrogen: CO molar ratio is ≤4, such as in the range of from 1 to 4.

In certain aspects, (i) the C₁₊ inorganic oxygenate comprises CO and (ii) the CO is obtained from syngas. The syngas can comprise, e.g., molecular hydrogen and ≥5.0 wt. % of carbon monoxide, based on the weight of the syngas. The syngas can have an H₂:(CO+CO₂) molar ratio in the range of from 0.5 to 20, e.g., in the range of from 0.6 to 4, such as an H₂:CO molar ratio in the range of from 0.6 to 4. The syngas can be produced by any convenient method, including conventional methods such as those specified in connection with feeds containing C₁₊ organic oxygenate.

Another suitable source of methane and co-feed is product obtained from the oxidative conversion of methane to ethylene and higher hydrocarbon (“OCM”). OCM is a process in which methane is reacted with an oxygen-containing gas in the presence of an OCM catalyst. The reaction is described in, for example, U.S. Pat. No. 5,336,825, the entire contents of which are incorporated herein by reference. The process couples the methane into higher hydrocarbon, such as ethylene, by reactions such as:

2CH₄+O₂→C₂H₄+2H₂O.

In the OCM process, methane is activated heterogeneously on the OCM catalyst surface, forming methyl free radicals, which then couple in the gas phase to form ethane, which subsequently undergoes dehydrogenation to form ethylene. Since OCM product has the desirable feature that it further comprises C₂₊ aliphatic hydrocarbon and unconverted methane, an OCM product can be utilized directly as a combined feed for the specified methane conversion reaction. For example, a combined feed consisting essentially of or even consisting of OCM product can be fed to the specified methane conversion reaction. Alternatively, the combined feed can comprise OCM product and one or more of added methane, added C₁₊ inorganic oxygenate, added molecular hydrogen, added C₂₊ hydrocarbon, and added diluent.

Suitable sources of co-feed, and optionally the combined feed, also include a reaction product obtained from hydrocarbon pyrolysis. For example, a combined feed obtained from a reaction product produced in the pyrolysis of methane and/or ethane can be fed to the specified methane conversion reaction. Optionally, the combined feed comprises such a pyrolysis product, and further comprises one or more of added methane, added C₁₊ inorganic oxygenate, added molecular hydrogen, and/or added C₂₊ hydrocarbon.

In certain aspects, the co-feed is methanol produced from natural gas via syngas. The syngas can comprise, e.g., molecular hydrogen and ≥5.0 wt. % of carbon monoxide, based on the weight of the syngas, and the syngas can have an H₂: (CO+CO₂) molar ratio in the range of from 0.5 to 20, e.g., an H₂: CO molar ratio in the range of from 0.5 to 20, e.g., 0.6 to 4.

When syngas is a direct source of inorganic oxygenate and/or an indirect source of organic oxygenate, the syngas can be produced by any convenient method, including conventional methods such as the partial oxidation of methane and/or the steam reforming of methane. Suitable methods include those described in U.S. Publication Nos. 2007/0259972 A1, 2008/0033218 A1, and 2005/0107481, each of which is incorporated by reference herein in its entirety.

When the desired co-feed includes organic alcohol produced from syngas, the alcohol can be produced by conventional processes, but the invention is not limited thereto. For example, the alcohol (such as methanol) can be produced from syngas at very high selectivity using a mixture of copper, zinc oxide, and alumina at a temperature of 200° C. to 400° C. and pressures of 50-500 atm. In addition to Cu/ZnO/Al₂O₃, other catalyst systems suitable for methanol synthesis include Zn/VCr₂O₃, Cu/ZnO, Cu/ZnO/Cr₂O₃, Cu/ThO₂, CoSₓ, MoSₓ, Co—MoSₓ, Ni—Sₓ, Ni—MoSₓ, and Ni—Co—MoSₓ.

Aspects of the invention which include contacting the specified combined feed with the conversion catalyst to produce C₅₊ hydrocarbon, e.g., aromatics, such as BTX will now be described in more detail. The invention is not limited to these aspects, and this description is not meant to foreclose other aspects within the broader scope of the invention, such as those which are carried out by separately conveying methane and the co-feed to the conversion catalyst.

### Converting the Feed to CS₊ Hydrocarbon

The reacting of the methane in the presence of the specified co-feed and conversion catalyst to produce the desired products can be carried out over a broad range of process conditions and methane: co-feed ratios. Although the mechanisms of the reactions occurring in the present process are not fully understood, and not wishing to be bound by any theory or model, it is believed that the methane is activated proximate to the specified active metal located in pores of the molecular sieve, and particularly in pores having openings through the molecular sieve's outer surface, It is also believed that locating ≥90 wt. % of the active metal in the pores of the conversion catalyst's molecular sieve, with ≤10 wt. % of the active metal located proximate to the molecular sieve's outer surface, undesired methanol conversion side-reactions are substantially avoided even at elevated temperature. This in turn leads to an increase in methanol available for alkylating aromatic hydrocarbon formed in the pores. This beneficially results in appreciable yields of toluene and xylenes. It is also believed that since specified conversion catalyst contains pores having an average pore size of 4 Å to 7 Å, the conversion reaction favors the incorporation of activated methane fragments into hydrocarbon compounds having 5-10 carbon atoms. This in turn leads to a decrease in the yield of hydrocarbon compounds having more than 10 carbon atoms, resulting in less coke yield than is the case with conventional methane conversion processes. Advantageously, low coke yield is achieved even when the conversion conditions include a relatively high temperature, e.g., at a temperature ≥500° C., e.g., ≥600° C., such as ≥700° C., or ≥800° C.

Particularly when the co-feed includes C₁₊ inorganic oxygenate and/or C₁₊ organic oxygenate, it can also be beneficial to carry out the conversion at a temperature <500° C., e.g., ≤450° C., such as ≤400° C., or ≤350° C., or even ≤300° C. Whereas the non-oxidative methane conversion is thermodynamically restrained at low temperatures and the conversion of C₁₊ oxygenates is highly exothermic, the specified conversion couples the simultaneous endothermic conversion of methane and the exothermic aromatization of the C₁₊ oxygenate, rendering the process highly energy efficient. This effect, when combined with the conversion synergies resulting from locating the dehydrogenation metal sites and the acid sites primarily in the pores of the conversion catalyst's molecular sieve, leads to an increased yield of the desired C₅₊ hydrocarbon products, as compared with conventional methane conversion processes operating under similar process conditions.

Certain conversion catalysts within the scope of the invention will now be described in more detail. The invention is not limited to these conversion catalysts, and this description is not meant to foreclose other conversion catalysts within the broader scope of the invention.

### Representative Conversion Catalysts

The conversion catalyst comprises at least one molecular sieve and at least one active metal. The active metal can be, e.g., in the form of one or more neutral metals, such as those selected from Groups 3 to 13 of the Periodic Table. The conversion catalyst's molecular sieve has a framework of interconnected atoms. The framework defines an outer surface of the molecular sieve and a plurality of pores located within the molecular sieve. A feature of the conversion catalyst's molecular sieve is that at least some of the molecular sieve's pores have an average pore size in the range of from 4 Å to 7 Å. Typically, the pores are not completely enclosed by the molecular sieve's outer surface. Instead, at least some of the pores typically have one or more pore openings through the outer surface. Another feature of the molecular sieve is that ≥90 wt. % of the active metal is located in those pores having an average pore size in the range of from 4 Å to 7 Å, e.g., ≥95 wt. %, such as ≥99 wt. %, or ≥99.9 wt. %. Yet another feature of the molecular sieve is that ≤10 wt. % of the active metal is located proximate to the outer surface, e.g., ≤5 wt. %, such as ≤1 wt. %, or ≤0.1 wt. %. More typically, the molecular sieve has at least one set of pores of substantially uniform size extending through the molecular sieve, wherein geometric mean of the cross-sectional dimensions of each of the pores is >4 Å, or >5 Å, or >5.3 Å, e.g., ≥5.4 Å such as ≥5.5 Å, or in the range of 5 Å to 7 Å, or 5.4 Å to 7 Å.

Typically, the conversion catalyst includes ≥10 wt. % of one or more molecular sieves and ≥0.005 wt. % of one or more of the active metals, e.g., ≥0.005 wt. % of one or more of the carbidic metals, wherein the molecular sieve has a Constraint Index in the range of from 1-12, e.g., 2-11. When the molecular sieve and active metal together constitute less than 100 wt. % of the conversion catalyst, ≥90 wt. % of the remainder of the conversion catalyst can include a matrix, such as ≥99 wt. % of the remainder. A feature of the conversion catalyst is that it includes ≤10 wt. % of metal in any form other than (A) framework metal if any (e.g., framework aluminum atoms) and (B) the active metal located in the pores of the molecular sieve. Since the active metal is primarily (e.g., more than 50 wt. %, such as more than 75 wt. %, or more than 90 wt. %) located in the pores of the molecular sieve, the conversion catalyst can be referred to as an “embedded catalyst”. When the molecular sieve is a silicoaluminate such as ZSM-5, e.g., ZSM-5 in hydrogen form (“HZSM-5”), the embedded catalyst can be represented symbolically by [metal]@ZSM-5, [metal]@HZSM-5, and the like. For example, when the active metal is molybdenum and the molecular sieve is HZSM-5, the embedded catalyst can be represented symbolically by Mo@HZSM-5. This symbolic representation is different from that used to represent conventional metal-impregnated ZSM-5, e.g., Mo-impregnated ZSM-5, which typically has the symbolic representation Mo/ZSSM-5 or Mo-ZSM-5.

The conversion catalyst typically includes the molecular sieve in an amount ≥20 wt. %, based on the weight of the conversion catalyst, e.g., ≥25 wt. %, such as ≥50 wt. %, or in the range of from 30 wt. % to 99.9 wt. %. The molecular sieve can includes aluminosilicate, e.g., ≥90 wt. % of at least one aluminosilicate. Zeolite is a suitable aluminosilicate, e.g., the molecular sieve can comprise ≥90 wt. % of at least one zeolite based on the weight of the molecular sieve, such as ≥95 wt. %, or ≥99 wt. %. The aluminosilicate, e.g., the zeolite, can be an un-substituted aluminosilicate, a substituted aluminosilicate, or a combination thereof, but is typically un-substituted.

The zeolite can be in hydrogen form, e.g., zeolite synthesized in the alkali metal form and then converted to the hydrogen form. This can be accomplished by exposing the zeolite to an exchange agent, e.g., ion of ammonium (typically in a solution, such as one or more of ammonium halide, ammonium nitrate, etc. Examples of suitable zeolites include ZSM-5 (including H-ZSM-5), ZSM-11, ZSM-12, ZSM-22, ZSM-23, ZSM-35, and ZSM-48, including mixtures and intermediates thereof such as ZSM-5/ZSM-11 admixture. For example, the molecular sieve can comprise ≥90 wt. % of (A) ZSM-5 and/or (B) ZSM-12, based on the weight of the molecular sieve, e.g., ≥95 wt. % of ZSM-5, such as ≥99 wt. % ZSM-5. In certain aspects, the molecular sieve has a relatively small crystal size, e.g., small crystal ZSM-5, meaning ZSM-5 having a crystal size ≤0.05 micrometers (μm), such as in the range of 0.02 μm to 0.05 μm. Small crystal ZSM-5 and the method for determining molecular sieve crystal size are disclosed in U.S. Pat. No. 6,670,517, which is incorporated by reference herein in its entirety.

Alternatively or in addition, the molecular sieve includes one or more of the MCM-22 family (including mixtures of MCM-22 family molecular sieve), e.g., MCM-22 alone or in combination with other molecular sieve such as one or more of the specified zeolites. The MCM-22 family includes those molecular sieves having an X-ray diffraction pattern including d-spacing maxima (in A) at 12.4±0.25, 6.9±0.15, 3.57±0.07 and 3.42±0.07. Examples of suitable MCM-22-family molecular sieve include PSH-3, SSZ-25, ITQ-1, MCM-36, MCM-49, MCM-56, UZM-8, ERB-1, and ITQ-2.

When the molecular sieve includes at least one aluminosilicate, the aluminosilicate's silica:alumina ratio (substantially the same as the aluminosilicate's Si:Al₂ atomic ratio) is typically ≥2, e.g., in the range of from 5 to 100. The silica:alumina ratio is meant to represent the Si:Al₂ atomic ratio in the rigid anionic framework of the crystalline aluminosilicate. It is within the scope of the invention to increase the conversion catalyst's resistance to deactivation (and to increase aromatic hydrocarbon yield) by including phosphorous as a conversion catalyst constituent, typically with the molecular sieve. When used, the amount of phosphorous is typically ≥1 wt. % based on the weight of the molecular sieve component. For example, when the molecular sieve component includes aluminosilicate, the phosphorous:aluminum atomic ratio can be in the range of from 0.01 to 1. Zeolite having a higher silica:alumina ratio can be utilized when a lower catalyst acidity is desired, e.g., in the range of from 44 to 100, such as from 50 to 80, or 55 to 75. When the conversion catalyst includes aluminosilicate which includes phosphorous, the phosphorous:aluminum atomic ratio is typically in the range of from 0.01 to 0.5. For example, the conversion catalyst can contain ≥10 wt. % of phosphorous-modified alumina, such as ≥15 wt. %, or in the range of from 10 wt. % to 20 wt. %.

In addition to the molecular sieve component, the conversion catalyst includes at least one active metal in amount ≥0.005 wt. %, based on the weight of the conversion catalyst. The active metal can include one or more neutral metals selected from Groups 3 to 13 of the Periodic Table, such as one or more of Ga, In, Zn, Cu, Re, Mo, W, La, Fe, Ag, Pt, and Pd, and/or one or more oxides, sulfides and/or carbides of these metals. For example, the active metal can be one or more of Mo, Ga, Zn. The active metal can be, e.g., one or more carbidic molybdenum compounds, such as one or more molybdenum carbides and/or one or more molybdenum oxycarbides.

Typically, the conversion catalyst includes ≥0.01 wt. % of the active metal, e.g., ≥0.1 wt. %, such as ≥0.5 wt. %, or ≥1 wt. %, or ≥5 wt. %, or ≥10 wt. %. These weight percents represent the weight of the metal, not the weight of the active-metal compound containing the active metal. For example, the conversion catalyst can comprise ≥1 wt. % of Mo in the form of MoO₃, such as ≥5 wt. %, or ≥10 wt. %. In certain aspects, (i) ≥99 wt. % of the active metal is one or more of Ga, Zn, Mo, and In, e.g., ≥99 wt. % of Mo in the form of one or more carbidic molybdenum compounds, such as one or 99 wt. % of Mo in the form of more molybdenum carbides and/or one or more molybdenum oxycarbides, and (ii) ≥99 wt. % of the molecular sieve is ZSM-5-type zeolite.

Besides the molecular sieve and active metal, the conversion catalyst can further include an optional matrix component (“matrix”), e.g., one or more inorganic binders. When the matrix includes metal, such as aluminum in a matrix comprising alumina, such metal is referred to as “matrix metal”. The amount of matrix is not critical. When present, the amount of matrix is typically in the range of 0.01 times the weight of the molecular sieve component to about 0.9 times the weight of the molecular sieve component, e.g., in the range of 0.02 to 0.8. The matrix can include active materials, such as synthetic or naturally occurring zeolites. Alternatively, or in addition, the matrix can include clays and/or oxides such as alumina, silica, silica-alumina, zirconia, titania, magnesia or mixtures of these and other oxides. The matrix can include naturally occurring materials and/or materials in the form of gelatinous precipitates or gels including mixtures of silica and metal oxides. Clays may also be included with the oxide type binders to modify the mechanical properties of the conversion catalyst or to assist in its manufacture.

Alternatively or in addition, the matrix can include one or more substantially inactive materials. Inactive materials suitably serve as diluents to control the amount of conversion so that products may be obtained economically and orderly without employing other means for controlling the rate of reaction. For example, besides any phosphorous that may be added to or impregnated into the molecular sieve, the matrix can optionally include phosphorous, e.g., to lessen catalyst acidity. Suitable phosphorous-containing matrices are disclosed in U.S. Pat. No. 5,026,937, which is incorporated by reference herein in its entirety. The matrix is optional. In certain aspects, the conversion catalyst is substantially-free of matrix, e.g., contains ≤1 wt. % of matrix, such as ≤0.1 wt. %. In particular, the conversion catalyst can be substantially free of binder, e.g., contains ≤1 wt. % of binder, such as ≤0.1 wt. %. For example, ≥95 wt. % of the conversion catalyst's molecular sieve can be self-bound bound molecular sieve, e.g., ≥95 wt. % can be self-bound ZSM-5, and in particular self-bound, small crystal ZSM-5.

The specified conversion catalysts can have any convenient form that is useful in the specified methane conversion reaction. For example, the conversion catalyst can have the form of a particulate, e.g., a plurality of catalyst particles having an average size ≤250 μm, e.g., in the range of 20 μm to 200 μm, and an average density in the range of from 0.6 g/cm³ to 2 g/cm³, e.g., in the range of from 0.9 g/cm³ to 1.6 g/cm³. Typically, the conversion catalyst has a surface area, as measured by nitrogen physisorption, in the range of from 100 m²/g to 600 m²/g, e.g., in the range of from 200 m²/g to 500 m²/g. The conversion catalyst can be located in one or more bed configurations, e.g., conventional bed configurations such as fixed bed, moving bed, ebullating bed, fluidized bed, etc. Any convenient reactor configuration can be used that is suitable for contacting the specified conversion catalyst with methane and the specified co-feed under the specified conversion conditions for an average residence time in the reaction zone that is sufficient for producing the desired C₅₊ hydrocarbon product. Conventional reactors are suitable, e.g., tubular reactors, including reverse-flow regenerative reactors, fluid-bed reactors, riser reactors, fixed bed reactors, etc. Any convenient method for controlling the average residence time in the reaction zone of the specified conversion catalyst under the specified conversion conditions can be used, including conventional methods for doing so, e.g., adding and removing conversion catalyst from a fluidized bed via inlet and outlet conduits, conveying the conversion catalyst through a riser reactor, operating flow control valves to regulate the flow of feed and regenerating fluid through a tube reactor containing the conversion catalyst, etc.

In certain aspects, the specified conversion catalyst comprises an active metal and molecular sieve, wherein (i) ≥99 wt. % of the active metal is Mo, and (ii) ≥99 wt. % of the molecular sieve is ZSM-5-type zeolite. Aspects of the invention relating to the synthesis of this conversion catalyst will now be described in more detail. The invention is not limited to these aspects, and this description is not meant to foreclose other aspects in the broader scope of the invention, such as aspects relating to other forms of the specified conversion catalyst.

### Catalyst Synthesis

The conversion catalyst is produced from the specified synthesis mixture under hydrothermal synthesis conditions. Conventional hydrothermal synthesis conditions can be used, but the invention is not limited thereto. The synthesis mixture comprises water, an aluminum source, a templating agent, and substantially one source of silicon. Typically, the synthesis mixture includes nitrate of aluminum and/or aluminate of at least one alkali metal; hydroxide, e.g., hydroxide of ammonium and/or hydroxide of at least one second alkali metal; water; oxygenate, such as alcohol, e.g., ethanol; and a templating agent such as TPABr, TPAOH, etc. A feature of the specified synthesis mixture is that it further comprises a constituent that is substantially the synthesis mixture's sole source of silicon. In certain aspects, (i) ≥90 wt. % of the silica source comprises SiO₂ impregnated with oxide of at least one metal selected from Groups 3 to 13 of the Periodic Table, e.g., ≥95 wt. %, such as ≥99 wt. %, or ≥99.9 wt. %, and (ii) the combination of the aluminum source(s), any hydroxide, the water, any alcohol, the templating agent, and any other components of the synthesis mixture does not contain an appreciable amount of silicon or compounds thereof. In this context, less than an appreciable amount means that there is ≤0.1 wt. % of silicon (including silicon present in silicon-containing compounds) in the combination of the aluminum source, any hydroxide, any alcohol, the water, the templating agent, and any other components of the synthesis mixture, e.g., ≤0.01 wt. %, such as ≤0.001 wt. %, or ≤0.0001 wt. %.

The silica source typically comprises a siliceous material and one or more metals selected from Groups 3 to 13 of the Periodic Table. The metal or metals are typically in the form of one or more metal compounds, e.g., one or more metal oxides. Suitable siliceous materials include porous SiO₂. Typically, the metal is located on, in, or proximate to the siliceous material, e.g., is located on, in, or proximate to the siliceous material. The term “located on or in” includes, e.g., forms in which the metals and/or compounds thereof are bound to the siliceous material, e.g., via one or more chemical bonds, such as via one or more hydrogen bonds. For example, suitable silica sources include MoO₃ and/or WO₃ bound via one or more hydrogen bonds to porous SiO₂, including those bound via one or more OH groups which are themselves bound to the SiO₂. Conventional methods can be used to prepare the silica source, but the invention is not limited thereto. Suitable conventional methods of locating the metal, metals, or compounds thereof, on, in, or proximate to the siliceous material include impregnation, incipient wetness, co-precipitation, evaporation, spray drying, sol-gel synthesis, ion exchange, chemical vapor deposition, diffusion, and even physical mixing of precursor species in an appropriate chemical state. When impregnation is used, e.g., the impregnation of MoO₃ into porous SiO₂, this can be carried out by contacting the porous SiO₂ with a solution comprising one or more molybdenum salts, such as ammonium hexamolybdate. Impregnated SiO₂ when used, is typically dried, and optionally calcined, before it is added to the synthesis solution.

Convention templating agents can be used for directing the structure of synthesis product toward the desired molecular sieve, but the invention is not limited thereto. When the desired molecular sieve includes ZSM-5, the templating agent can include, e.g., one or more of TPAOH, TPABr, etc. Hydroxide, e.g., ammonium hydroxide, is typically used to regulate to synthesis mixture's pH (e.g., its basicity) so that the silica source is gradually dissolved over the course of the synthesis time. While not wishing to be bound by any theory or model, it is believed that particulate formed during synthesis from metal contained in the silica source, e.g., particulate of MoO₃, serve as a substrate for nucleating the desired molecular sieve.

One typical conversion catalyst selected from the specified conversion catalysts comprises ZSM-5, e.g., HZSM-5, and at least one of (i) molybdenum carbide and (ii) molybdenum oxycarbide (“Catalyst A”). When producing Catalyst A, the synthesis mixture typically comprises, consists essentially of, or even consists of an aluminum source comprising nitrate of aluminum and/or aluminate of at least one alkali metal; hydroxide comprising hydroxide of ammonium and/or hydroxide of at least one second alkali metal; water; ethanol; a templating agent such as TPAOH; and one source of silicon, wherein (i) ≥90 wt. % of the silica source comprises SiO₂ impregnated with MoO₃, e.g., ≥95 wt. %, such as ≥99 wt. %, or ≥99.9 wt. %, and (ii) the aluminum source, the hydroxide, the water, the ethanol, the templating agent, and other components of the synthesis mixture if any together comprise ≤0.1 wt. % of oxide of silicon, e.g., ≤0.01 wt. %, such as ≤0.001 wt. %, or ≤0.0001 wt. %. Except for the features that (i) they do not in combination contain an appreciable amount of silicon or compounds thereof, and (ii) they direct the molecular sieve structure during hydrothermal synthesis to the desired structure, e.g., to ZSM-5, the choices of the aluminum source, the hydroxide, the templating agent, the alcohol source, and the source of the water are not critical. For example, the aluminum source is typically aluminum nitrate. Although the hydroxide is typically ammonium hydroxide, the hydroxide can alternatively or in addition include hydroxide of at least one second alkali metal, e.g., one havingthe formula M′OH, where M′ is Na and/or K. Optionally, M is not the same as M′, but typically they are the substantially the same alkali metal. For Catalyst A, the templating agent is typically tetrapropylammonium OH (“TPAOH”).

Those skilled in the art will appreciate that the relative amounts of the aluminum source, the hydroxide, the templating agent, the sole silicon source, the ethanol, and the water are selected to gradually dissolve the silica source and to achieve the desired stoichiometry of the desired molecular sieve and active metal. For example, for Catalyst A the synthesis mixture typically comprises per mole of Al(NO₃)₃: 10 to 60 moles of MoO₃-impregnated SiO₂, such as about 40 moles; 100 moles to 500 moles of NH₃OH, such as about 300 moles; 10 moles to 50 moles of TPAOH, such as about 30 moles; 50 moles to 200 moles of ethanol, e.g., about 100 moles; and 100 moles to 3000 moles of water, such as about 1000 moles.

When the desired conversion catalyst is Catalyst A, the catalyst synthesis can be carried out as follows. The aluminum source is dissolved in a mixture of the water and the ethanol. This solution is agitated (e.g., stirred) while adding the silica source. The templating agent is gradually added (e.g., drop-wise). This precursor mixture is agitated more vigorously (e.g., stirred more vigorously) at a temperate of about 25° C. for about four hours to about eight hours, e.g., about six hours. Afterward, the hydroxide is added to the stirred mixture, followed by 10 minutes to 60 minutes of additional stirring, e.g., about 30 minutes to produce the synthesis mixture. Any suitable vessel can be used for preparing the synthesis mixture, e.g., an autoclave. The synthesis mixture is maintained at a temperature in the range of from 170° C. to 190° C., e.g., about 180° C. under effective hydrothermal synthesis conditions for a time sufficient to form the Catalyst A, typical a time in the range of about 90 hours to about 150 hours, typically about 120 hours. The hydrothermal synthesis can be carried out in a Teflon-lines, stainless steel autoclave, for example. When the metal of the silica source is Mo, the resulting Catalyst A precursor is typically is in the form of crystalline Mo—ZSM-5 having a long axis of at least 1×10⁻⁶ meters. By virtue of its larger size, the Catalyst A precursor can be readily separated from other products of the hydrothermal synthesis such as analcime and α-quartz, although this is not required.

The catalyst precursor is activated to produce the specified conversion catalyst, e.g., to produce Catalyst A. The term “activation” means at least converting into catalytically active form, e.g., into carbidic form, at least a portion of those metals or metal oxides in the catalyst precursor which are derived from the silica source. Activation typically includes drying the catalyst precursor, calcining the dried precursor, and carburizing the calcined catalyst precursor. One or more optional treatments, e.g., to substitute hydrogen atoms for at least a portion of any alkali metal atoms present in the conversion catalyst or catalyst precursor can be carried out if desired. Those skilled in the art will appreciate that conventional drying, calcining, carburizing, and hydrogen substitution can be used, but the invention is not limited thereto. Suitable procedures described in U.S. Pat. Nos. 7,728,186 and 8,841,227, the specifications of which are incorporated by reference herein in their entireties. The following procedures are typical for producing Catalyst A from the Catalyst A precursor.

Drying can be carried out by exposing the catalyst precursor to a substantially non-reacting environment at the drying temperature, e.g., a temperature in the range of about 110° C. to 150° C. for a time in the range of from 8 hours to 16 hours, e.g., about 12 hours. The non-reacting environment can be established by providing a flow of a drying gas, e.g., air, molecular nitrogen, etc. Calcining can be carried out by espousing the catalyst to an oxidizing environment such as air and exposing the catalyst precursor to a gradually increasing the temperature. For example, the dried catalyst precursor can be exposed to an temperature which increases from 25° C. at a rate of about 0.5° C. to 5° C. per minute until a temperature of 550° C. is achieved. After holding this temperature substantially constant for a time sufficient to remove any remaining templating agent from the molecular sieve, typically from 1 hour to about 10 hours, the temperature is gradually decreased at a rate of about 0.5° C. to 5° C. per minute until a desired temperature (e.g., ambient temperature) is achieved.

Carburization can be carried out by exposing the calcined precursor to a gradually increasing temperature (e.g., at a rate of a about 5° C. per minute) in the presence of a flow of inert gas, e.g., molecular nitrogen and/or argon, until a temperature of about 550° C. is achieved. With the temperature at about 550° C., a flow of a carburizing gas, e.g., methane or a methane-molecular hydrogen mixture, is substituted for at least a portion of the inert gas flow. The temperature is again increased, e.g., at a rate of about 10° C. per minute until a temperature of about 650° C. is achieved. The carburizing precursor is exposed to this temperature of at least about ten minutes to produce the conversion catalyst, e.g., Catalyst A.

The conversion catalyst and/or precursor thereof can be subjected to one or more treatments. For example, at least a portion of any alkali metal (e.g., Na) on Catalyst A can be removed by contacting Catalyst A and/or precursor thereof with an ammonium halide, e.g., NH₄Cl. Other treatments include selectivation treatment to increase selectivity for producing desired aromatic hydrocarbon compounds such as para-xylene. For example, the selectivation can be carried out before introduction of the conversion catalyst into the reactor and/or in-situ in the reactor, e.g., by contacting the conversion catalyst with a selectivating agent, such as at least one organosilicon compound, typically with a liquid carrier and subsequently calcining the conversion catalyst at a temperature of 350° C. to 550° C. This selectivation procedure can be repeated two or more times and alters the diffusion characteristics of the conversion catalyst such that the formation of para-xylene over other xylene isomers is favored. Such a selectivation process is described in detail in U.S. Pat. Nos. 5,633,417 and 5,675,047.

In certain aspects, the invention relates to contacting methane with at least one member of the class of specified co-feed in the presence of at least one member of the class of specified conversion catalysts, e.g., Catalyst A. Selected process conditions for the conversion will now be described in more detail. The invention is not limited to these process conditions, and this description is not meant to foreclose other process conditions within the broader scope of the invention.

### Process Conditions

In certain aspects, conversion of the feed comprising methane and co-feed to aromatic hydrocarbon is generally conducted at a temperature ≤1200° C. and a pressure in the range of from 0.01 bar (absolute) to 5 bar (absolute) (1 to 500 kPa absolute). Space velocity is not critical, and when the co-feed is in the vapor phase during the conversion the gas hourly space velocity is typically ≥1 cm³/h/g of conversion catalyst. The conversion process can be conducted in one or more fixed bed, moving bed or fluidized bed reaction zones. The conversion can be operated, e.g., continuously, semi-continuously, or in batch mode.

In certain aspects, the conversion is carried out in one or more reaction zones. Since the conversion reaction is typically net endothermic, the temperature in each reaction zone containing the conversion catalyst will tend to decrease from a maximum temperature to a minimum temperature as the reaction proceeds. Suitable conversion conditions in each reaction zone typically include (i) a maximum temperature of ≤1200° C., e.g., in the range of about 700° C. to about 1200° C., such as about 800° C. to about 950° C., and (ii) a minimum temperature ≥250° C., e.g., of about 400° C. to about 800° C., such as about 500° C. to about 700° C. For example, the reactor can have an average temperature in the range of from 400° C. to 800° C., e.g., 450° C. to 800° C., such as from 450° C. to 700° C. The term “average temperature” means the arithmetic mean of reactor temperature at the reactor inlet, the reactor outlet, and at a plurality of substantially evenly-spaced locations along the average flow direction between the inlet and outlet. The invention is compatible with the conventional practice of supplying heat to the conversion reaction to reduce the temperature drop across regions of the reaction zones and across the reactor during the reaction, e.g., to a temperature drop of essentially zero. The invention is also compatible with the conventional practice of establishing an inverse temperature profile across the reactor, e.g., by establishing a flow of heated fluidized conversion catalyst to one or more of the reaction zones. Doing so can achieve a temperature difference between the reactor product outlet and methane inlet of at least +10° C., such as at least +50° C., for example at least +100° C., and even at least +150° C.

In aspects where the conversion catalyst is in the form of a flowing particulate, the conversion catalyst typically enters the conversion reactor at a first, high temperature, e.g., in the range of about 800° C. to about 1200° C., such as about 900° C. to about 1100° C., and exits the reaction system at a second lower temperature, e.g., in the range of about 500° C. to about 800° C., such as about 600° C. to about 700° C. The total temperature difference of the catalytic particulate material across the reaction zones is generally at least 100° C.

Other conditions used in the methane conversion reaction generally include a pressure of about 1 kPa to about 1000 kPa, such as about 10 kPa to about 500 kPa, for example about 50 kPa to about 200 kPa and a weight hourly space velocity of about 0.01 to about 1000 hr⁻¹, such as about 0.1 to about 500 hr⁻¹, for example about 1 to about 20 hr⁻¹. The conversion is typically conducted in the absence of O₂, e.g., to prevent undesired OCM side-reactions, although other inorganic oxygenate may be present in the co-feed.

When it is desired to increase the amount of BTX in the C₅₊ hydrocarbon, particularly when the co-feed is a oxygenate, e.g., increasing the relative amount of toluene and xylenes, such as increasing the relative amount of paraxylene, the conversion conditions can include one or more of the following features: a temperature in the range of 400° C. to 800° C., e.g., 450° C. to 800° C., such as from 450° C. and 700° C., or from 550° C. to about 650° C.; a pressure between 14 psig and 1000 psig (between 100 kPa and 7000 kPa), or between 10 psig and 200 psig (between 170 kPa and 1480 kPa); a weight hourly space velocity (“WHSV”) for total hydrocarbon feed, typically the methane and any hydrocarbon in the co-feed, in the range of from 0.2 to 1000 hr⁻¹, and a WHSV for the C₁₊ oxygenate, e.g., methanol, in the range 0.01 to 100 hr⁻¹, based on total weight of conversion catalyst. Process conditions which lead to the production of an appreciable amount of aromatic hydrocarbon in the C₅₊ hydrocarbon product are referred to as “dehydrocyclization” process conditions.

When the conversion is carried out under ddehydrocyclization conditions, aromatic hydrocarbon is produced by removing hydrogen and cyclizing a non-cyclic hydrocarbon, e.g., one or more of the methane, the C₂₊ hydrocarbon in the co-feed, and hydrocarbon fragments produced by cracking the C₁₊ oxygenate co-feed. Besides aromatics, dehydrocyclization conditions can also produce an appreciable amount of non-aromatic hydrocarbon, e.g., (i) cyclo-paraffin and/or (ii) cyclo-olefin. Dehydrocyclization can be carried out in one step, in two steps, e.g., dehydrogenation followed by cyclization of the dehydrogenated intermediate; or in three or more steps, e.g., normal paraffin dehydrogenation, cyclization of the olefinic intermediate, and additional dehydrogenation (aromatization) of the cyclo-olefin intermediate. The dehydrocyclization (including any dehydrogenation carried out in connection with dehydrocyclization) is “non-oxidative” meaning that the reaction is carried out with little if any oxidative coupling of feed hydrocarbon, intermediate hydrocarbon (if any), or dehydrocyclization product.

A feature of the specified process is that it can be operated over a wide range of conversion conditions. Particularly in aspects where (i) the conversion catalyst is located in a fixed bed, and (ii) relatively long rung lengths are desired before catalyst regeneration or replacement is needed, such as more than four days, the conversion conditions can include one or more of the following features: a temperature in the range of from 275° C. to 650° C., e.g., 300° C. to 600° C., such as 325° C. to 550° C.; a pressure in the range of from 1.2 bar (abs) to 4 bar (abs), and a gas hourly space velocity of the methane+co-feed in the range of 100 cm³/h/g of conversion catalyst to 10,000 cm³/h/g of conversion catalyst, e.g., from 500 cm³/h/g to 5000 cm³/h/g.

When reacting methane and the specified co-feed in the presence of the specified conversion catalyst under the specified conversion conditions, the products of the conversion are mainly C₅₊ hydrocarbon, water, and lesser amounts of ethylene, ethane, propylene, propane and C₄ hydrocarbon. For example, the product of the conversion can comprise, e.g., (i) ≥5.0 wt. % of C₅₊ hydrocarbon, e.g., ≥10.0 wt. %, such as ≥15 wt. %; and (ii) ≤10.0 wt. % C₂ to C₄ hydrocarbon, e.g., 5.0 wt. %, such as ≤1.0 wt. %; the weight percents being based on the weight of the product. Methane conversion is generally ≥5.0 wt. %, based on the weight of methane in the feed, e.g., ≥10.0 wt. %, such as ≥15.0 wt. %.

The C₅₊ hydrocarbon comprises mainly aromatics, e.g., ≥50.0 wt. % of C₆ to C₁₀ aromatics, based on the weight of the product's C₅₊ hydrocarbon, such as ≥75.0 wt. %, or ≥90.0 wt. %, or ≥95.0 wt. %. For example, the molar ratio of aromatic hydrocarbon produced to methane converted is generally ≥3.5:1, such as ≥4:1.

The C₆ to C₁₀ aromatics can readily be removed from the product by any convenient method, e.g., by one or more conventional fractionation and extraction techniques.

### EXAMPLE

A sample of suitable Mo@HZSM-5 catalyst is prepared by a hydrothermal synthesis, using tetrapropylammonium hydroxide (TPAOH) as the templating (e.g., structure-directing) agent. MoO₃/SiO₂ is the substantially sole source of silicon. The aluminum source is Al(NO₃)₃.9H₂O. The pH (basicity) of the synthesis mixture is controlled by including sufficient NH₃.H₂O to dissolve the silica from MoO₃/SiO₂ over the course of the hydrothermal synthesis. The MoO₃ particles derived from the silica source serve as substrates for the nucleation and growth of HZSM-5 crystals with the templating agent tetrapropylammonium hydroxide (TPAOH). The molar ratio of the reactants in the synthesis mixture is 30 TPAOH: 100 ethanol: 1 Al(NO₃)₃: 40 SiO₂: 1000 H₂O: 300 NH₃. Initially, Al(NO₃)₃.9H₂O is dissolved in H₂O and ethanol in a Teflon-lined stainless steel autoclave, followed by the addition of the Mo/SiO₂ silica source (in the form of a powder) under stirring. Then TPAOH is added drop-wise, and afterwards, this solution is further vigorously stirred at a temperature of about 25° C. for about 6 hours. Ammonium hydroxide is added to the solution, which is stirred for about 30 minutes. Afterward, the autoclave is sealed and the synthesis is carried out in the autoclave at a temperature of about 453° K for about 120 hours. After cooling the autoclave naturally to about 300° K, the catalyst precursor is separated by filtration and washed with deionized water and ethanol several times. The catalyst precursor is then activated by drying in air at a temperature of about 393° K for about 12 hours, cooling the dried precursor to a temperature of about 300° K, and then calcining the dried precursor. Calcining is carried out by exposing the dried precursor to a temperature which increases from about 300° K to 823° K (in air) with a temperature ramping rate of 2° K·min⁻¹, and maintaining the precursor at 823° K for about 6 hours to remove any remaining organic material derived from the templating agent. The calcined precursor is carburized to produce the catalyst.

All patents, test procedures, and other documents cited herein, including priority documents, are fully incorporated by reference to the extent such disclosure is not inconsistent and for all jurisdictions in which such incorporation is permitted.

While the illustrative forms disclosed herein have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside herein, including all features which would be treated as equivalents thereof by those skilled in the art to which this disclosure pertains.

When numerical lower limits and numerical upper limits are listed herein, ranges from any lower limit to any upper limit are contemplated, and are expressly within the scope of the invention. The term “comprising” is synonymous with the term “including”. Likewise whenever a composition, an element or a group of components is preceded with the transitional phrase “comprising”, it is understood that we also contemplate the same composition or group of components with transitional phrases “consisting essentially of,” “consisting of”, “selected from the group of consisting of,” or “is” preceding the recitation of the composition, component, or components, and vice versa.

1. A process for producing C₅₊ hydrocarbon, the process comprising:
(a) providing methane and at least one co-feed comprising one or more of C₂₊ hydrocarbon, C₁₊ organic oxygenate, and inorganic oxygenate;
(b) providing a conversion catalyst comprising at least one molecular sieve and at least one active metal, wherein
(i) the molecular sieve has a framework of interconnected atoms,
(ii) the molecular sieve has an outer surface and a plurality of pores having an average pore size of 4 Å to 7 Å,
(iii) the active metal is selected from Groups 3 to 13 of the Periodic Table,
(iv) ≥90 wt. % of the active metal is located in the pores, and ≤10 wt. % of the active metal is proximate to the outer surface, and
(v) the conversion catalyst includes ≤10 wt. % of metal in any form other than (A) framework metal if any and (B) the active metal;
(c) contacting the methane and the co-feed with the conversion catalyst under conversion conditions which include a temperature <1200° C. to convert at least part of the methane and co-feed to a product comprising at least 5 wt. % of C₅₊ hydrocarbon based on the weight of the product; and
(d) separating at least part of the C₅₊ hydrocarbon from the product.

2. The process of claim 1, wherein ≥90 wt. % of the co-feed is organic alcohol.

3. The process of claim 1, wherein ≥90 wt. % of the co-feed is methanol.

4. The process of claim 1, wherein the methane and the co-feed are provided to step (c) at a methane: co-feed weight ratio in the range of from 2 to 100.

5. The process of claim 1, wherein
(i) the conversion catalyst includes the molecular sieve in an amount ≥10 wt. % of the conversion catalyst,
(ii) the molecular sieve comprises ≥90 wt. % of one or more of MCM-22, ZSM-5, ZSM-11, ZSM-12, ZSM-22, ZSM-23, ZSM-35, and ZSM-48,
(iii) the conversion catalyst includes the active metal in an amount ≥0.005 wt. % of the conversion catalyst, and
(iv) the active metal comprises ≥90 wt. % of one or more of Ga, Zn, Cu, Re, Mo, W, La, Fe, Ag, Pt, and Pd.

6. The process of claim 1, wherein the conversion conditions include a temperature in the range of from 275° C. to 1200° C., a pressure in the range of from 1 kPa to 10000 kPa, and a total space velocity (“Total WHSV”, based on the methane weight, the co-feed weight, and the conversion catalyst weight) in the range of from 0.01 hr⁻¹ to 1000 hr⁻¹.

7. The process of claim 1, wherein the temperature is in the range of from 450° C. to 800° C., a pressure in the range of from 100 kPa to 7000 kPa, and wherein the conversion conditions further include a hydrocarbon space velocity (“Hydrocarbon WHSV”, based on the methane weight, the weight of any hydrocarbon in the co-feed, and the conversion catalyst weight) in the range of from 0.2 hr⁻¹ to 1000 hr⁻¹, and/or a Ci oxygenate space velocity (“C₁₊ oxygenate WHSV”, based the weight of any Ci oxygenate in the co-feed and the weight of the conversion catalyst).

8. The process of claim 1, wherein the contacting conditions including one or more of a temperature in the range of from 275° C. to 450° C., a pressure in the range of from 120 kPa to 400 kPa (absolute), and a feed gas hourly space velocity of ≥100 cm³ of feed per gram of conversion catalyst per hour.

9. The process of claim 1, wherein ≥90 wt. % of molecular sieve is ZSM-5 and ≥90 wt. % of the active metal is Mo in carbidic form.

10. The process of claim 9, wherein (i) the ZSM-5 is produced by a hydrothermal reaction of a synthesis mixture, (ii) the synthesis mixture comprises water, ethanol, hydroxide, an aluminum source, a templating agent, and substantially one source of silicon, and (iii) ≥90 wt. % of the silicon source is MoO₃-impregnated SiO₂.

11. A natural gas upgrading process, comprising:
(a) providing a feed comprising a natural gas which comprises methane and at least one C₂₊ paraffinic hydrocarbon;
(b) providing a conversion catalyst comprising at least one molecular sieve and at least one active metal, wherein
(i) the molecular sieve has a framework of interconnected atoms,
(ii) the molecular sieve has an outer surface and a plurality of pores having an average pore size of 4 Å to 7 Å,
(iii) the active metal is selected from Groups 3 to 13 of the Periodic Table,
(iv) ≥90 wt. % of the active metal is located in the pores, and ≤10 wt. % of the carbidic metal is proximate to the outer surface, and
(v) the conversion catalyst includes ≤10 wt. % of metal in any form other than (A) the active metal, (B) framework metal if any, and (C) matrix metal if any;
(c) contacting the natural gas with the conversion catalyst under conversion conditions which include a temperature <1200° C. to convert at least part of the methane in the natural gas to a product comprising at least 5 wt. % of C₅₊ hydrocarbon based on the weight of the product; and
(d) separating at least part of the C₅₊ hydrocarbon from the product.

12. The process of claim 11, wherein the natural gas comprises 1 mole % to 95 mole % of methane, 5 mole % to 50 mole % of ethane, 2 mole % to 40 mole % of propane, 0.1 mole % to 30 mole % of i-butane, and 1 mole % to 30 mole % of n-butane, and 0.05 mole % to 25 mole % of i-pentane.

13. The process of claim 11, wherein the natural gas is an associated gas which contains CO₂.

14. The process of claim 11, wherein the natural gas contacts the conversion catalyst in the presence of at least one organic oxygenate.

15. The process of claim 11, wherein:
(i) the conversion catalyst includes the molecular sieve in an amount ≥10 wt. % of the conversion catalyst,
(ii) the molecular sieve comprises ≥90 wt. % of one or more of MCM-22, ZSM-5, ZSM-11, ZSM-12, ZSM-22, ZSM-23, ZSM-35, and ZSM-48,
(iii) the conversion catalyst includes the active metal in an amount ≥0.005 wt. % of the conversion catalyst, and
(iv) The active metal comprises ≥90 wt. % of one or more of Ga, Zn, Cu, Re, Mo, W, La, Fe, Ag, Pt, and Pd.

16. The process of claim 11, wherein the conversion conditions include a temperature in the range of from 275° C. to 1200° C., a pressure in the range of from 1 kPa to 10000 kPa, and a total space velocity (“Total WHSV”, based on the methane weight, the co-feed weight, and the conversion catalyst weight) in the range of from 0.01 hr⁻¹ to 1000 hr⁻¹.

17. The process of claim 11, wherein the temperature is in the range of from 450° C. to 800° C., a pressure in the range of from 100 kPa to 7000 kPa, and wherein the conversion conditions further include a hydrocarbon space velocity (“Hydrocarbon WHSV”, based on the weight of hydrocarbon in the natural gas and the conversion catalyst weight) in the range of from 0.2 hr⁻¹ to 1000 hr⁻¹, and/or a Ci oxygenate space velocity (“Ci oxygenate WHSV”, based the weight of any oxygenate in the co-feed and the weight of the conversion catalyst).

18. The process of 11, wherein the contacting conditions including one or more of a temperature in the range of from 275° C. to 450° C., a pressure in the range of from 120 kPa to 400 kPa (absolute), and a feed gas hourly space velocity of ≥100 cm³ of feed per gram of conversion catalyst per hour.

19. The process of claim 11, wherein (i) ≥90 wt. % of molecular sieve is ZSM-5, and (iii) ≥90 wt. % of the active metal is Mo in carbidic form.

20. The process of claim 19, wherein (i) the ZSM-5 is produced by a hydrothermal reaction of a synthesis mixture, (ii) the synthesis mixture comprises water, ethanol, hydroxide, an aluminum source, a templating agent, and substantially one source of silicon (iii) ≥90 wt. % of the silicon source is MoO₃-impregnated SiO₂.

21. A conversion product produced by a process comprising:
(a) providing a synthesis mixture comprising at least one aluminum source, water, a templating agent, and one silica source, wherein (i) ≥90 wt. % of the silica source comprises SiO₂ impregnated with at least one metal, or compound thereof, selected from Groups 3 to 13 of the Periodic Table and (ii) the aluminum source, the water, the templating agent, and other components of the synthesis mixture if any together comprise ≤0.1 wt. % of silicon;
(b) providing methane and at least one co-feed comprising one or more of C₂₊ hydrocarbon, C₁₊ organic oxygenate, and inorganic oxygenate;
(c) reacting the synthesis mixture under hydrothermal reaction conditions to produce a reaction product comprising molecular sieve;
(d) activating at least a portion of the molecular sieve to produce a conversion catalyst; and
(e) contacting the methane and the co-feed with at least a portion of the conversion catalyst under conversion conditions which include a temperature <1200° C. to convert at least part of the methane and co-feed to the conversion product, the conversion product comprising at least 5 wt. % of C₅₊ hydrocarbon.

22. The conversion product of claim 21, wherein (i) the aluminum source comprises aluminum nitrate and/or aluminate of at least one alkali metal, (ii) the synthesis mixture further comprises one or more of alcohol, hydroxide of ammonium, and hydroxide of at least one second alkali metal, and (iii) when synthesis mixture comprises the hydroxide of at least one second alkali metal the activation includes contacting the molecular sieve with ion of ammonium to remove at least a portion of any of the second alkali metal from the molecular sieve.

23. The conversion product of claim 21, wherein (i) the hydrothermal reaction conditions of step (c) include forming a gel from the synthesis mixture exposing the gel to a temperature of at least 150° C. for at least 24 hours, and the activation includes drying and calcining the molecular sieve.

24. The conversion product of claim 22, wherein:
(i) the silica source comprises SiO₂ impregnated with MoO₃, the aluminum source includes aluminum nitrate, the synthesis mixture includes ethanol and ammonium hydroxide, and the templating agent is TPAOH,
(ii) ≥90 wt. % of the co-feed is methanol, and
(iii) the conversion conditions of step (e) include a temperature is in the range of from 450° C. to 700° C., a pressure in the range of from 100 kPa to 7000 kPa, a hydrocarbon space velocity (“Hydrocarbon WHSV”, based on the methane weight, the weight of any hydrocarbon in the co-feed, and the conversion catalyst weight) in the range of from 0.2 hr⁻¹ to 1000 hr⁻¹, and a methanol space velocity (“Methanol WHSV”, based the weight of the methanol in the co-feed and the weight of the conversion catalyst).

25. The conversion product of claim 21, wherein the conversion product comprises ≥10 wt. % of aromatic hydrocarbon which includes paraxylene, and at least a portion of the paraxylene is separated from the conversion product during or after step (e).

26. A conversion product produced by a process comprising:
(a) providing a synthesis mixture comprising Al(NO₃)₃, ammonium hydroxide, TPAOH, water, ethanol, and one silica source, wherein (i) ≥90 wt. % of the silica source comprises SiO₂ impregnated with MoO₃ and (ii) Al(NO₃)₃, ammonium hydroxide, the TPAOH, the water, and other components of the synthesis mixture if any together comprise ≤0.1 wt. % of silicon;
(b) providing methane and at least one co-feed comprising one or more of C₂₊ hydrocarbon, C₁₊ organic oxygenate, and inorganic oxygenate;
(c) reacting the synthesis mixture under hydrothermal reaction conditions to produce a reaction product comprising molecular sieve;
(d) activating the molecular sieve to produce a conversion catalyst; and
(e) contacting the methane and the co-feed with the conversion catalyst under conversion conditions which include a temperature <1200° C. to convert at least part of the methane and co-feed to the conversion product, the conversion product comprising at least 5 wt. % of C₅₊ hydrocarbon.
